(19) United States
(12) Patent Application Publication
(10) Pub. No.: US 2022/0047226 A1
(43) Pub. Date: Feb. 17, 2022

(54) HEALTH ASSISTANCE SYSTEM AND WEARABLE DEVICE

(71) Applicant: FUJIKIN SOFT CO., LTD., Osaka (JP)

(72) Inventors: Shinya NOJIMA, Osaka (JP); Shuji NAKABAYASHI, Osaka (JP); Masashi KISHI, Osaka (JP); Hidenobu SATO, Osaka (JP)

(21) Appl. No.: 17/509,647

(22) Filed: Oct. 25, 2021

Related U.S. Application Data
(63) Continuation of application No. PCT/JP2020/015586, filed on Apr. 6, 2020.

(30) Foreign Application Priority Data
Apr. 24, 2019 (JP) 2019-082859

(51) Int. Cl. A61B 5/00; A61B 5/0205; G16H 20/00; G16H 40/67

(52) U.S. Cl. CPC A61B 5/7435; A61B 5/02055; G16H 40/67; G16H 20/00; A61B 5/681

(57) ABSTRACT

A health assistance system includes: a display portion; an obtaining portion that is configured to obtain a plurality of types of information on health of a user; and a health information creation portion that is configured to create health information on a health condition of the user, based on the information obtained by the obtaining portion, and cause the display portion to display the health information.

### CROSS REFERENCE TO RELATED APPLICATION

This application is a continuation under 35 U.S.C. § 120 of PCT/JP2020/015586, filed Apr. 6, 2020, which is incorporated herein reference and which claimed priority to Japanese Application No. 2019-082859, filed Apr. 24, 2019. The present application likewise claims priority under 35 U.S.C. § 119 to Japanese Application No. 2019-082859, filed Apr. 24, 2019, the entire content of which is also incorporated herein by reference.

### TECHNICAL FIELD

The present disclosure relates to a health assistance system and a wearable device.

### BACKGROUND

A technique has been proposed that obtains biological information, such as a user's blood pressure or the like, and displays the obtained biological information (see Japanese Patent Application Publication No. 2014-168492, for example).

### SUMMARY

With the technique of the Japanese Patent Application Publication No. 2014-168492, however, it is not possible to ascertain the user's health condition at a glance.

Thus, it is an object of the present disclosure to provide a health assistance system and a wearable device that allow a user to ascertain his or her health condition at a glance.

In order to attain the above object, a health assistance system according to one embodiment of the present disclosure includes: a display portion; an obtaining portion that is configured to obtain a plurality of types of information on health of a user; and a health information creation portion that is configured to create health information on a health condition of the user, based on the information obtained by the obtaining portion, and cause the display portion to display the health information.

A wearable device according to one embodiment of the present disclosure includes: a display portion; an obtaining portion that is configured to obtain a plurality of types of information on health of a user; and a health information creation portion that is configured to create health information on a health condition of the user based on the information obtained by the obtaining portion, and cause the display portion to display the health information.

### BRIEF DESCRIPTION OF THE DRAWINGS

In the drawings:

FIG. 1 illustrates a schematic view of a health assistance system according to an embodiment;

FIG. 2 illustrates a block diagram of a wearable device and a mobile terminal device of the health assistance system;

FIG. 3 illustrates a diagram of a radar chart regarding a user's health condition;

FIG. 4 illustrates a diagram of an icon regarding a user's health condition;

FIG. 5 illustrates an explanatory diagram of contents displayed on a display portion;

FIG. 6 illustrates an explanatory diagram of an improvement information table;

FIG. 7 illustrates a diagram of the appearance of a wearable device according to a modification;

FIG. 8 illustrates a block diagram of a wearable device and a mobile terminal device of a health assistance system according to a modification; and

FIG. 9 illustrates a diagram of a radar chart relating to a user's health condition according to a modification.

### DETAILED DESCRIPTION

Referring to the drawings, a health assistance system 1 according to an embodiment of the present disclosure is now described.

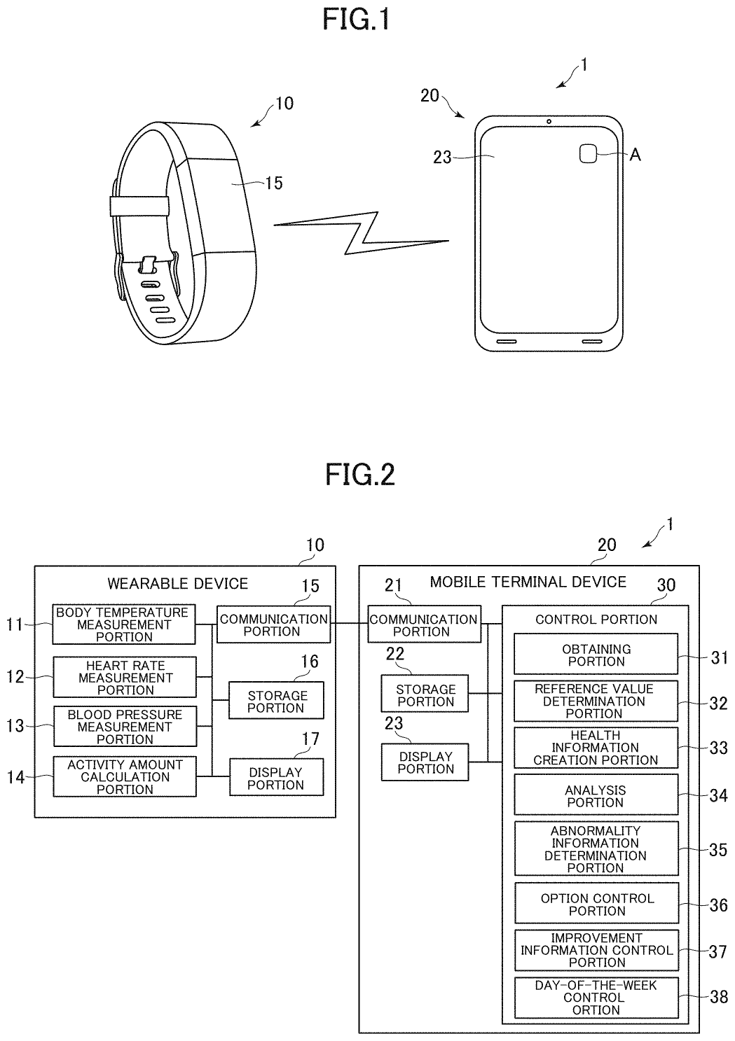

FIG. 1 illustrates a schematic view of the health assistance system 1 according to the present embodiment.

FIG. 2 illustrates a block diagram of a wearable device 10 and a mobile terminal device 20 of the health assistance system 1.

The health assistance system 1 according to the present embodiment includes the wristband-type wearable device 10 and the mobile terminal device 20, such as a smartphone. The wearable device 10 and the mobile terminal device 20 are connected via short-range wireless communication or the like to communicate information with each other. The wearable device 10 according to the present embodiment is not limited to a wristband-type device, such as a wristwatch, and may take various forms such as a clip, a belt, and smartglasses. Also, the mobile terminal device 20 may be various devices including a multifunctional mobile terminal, such as a tablet, and a personal computer.

The wearable device 10 includes a body temperature measurement portion 11, a heart rate measurement portion 12, a blood pressure measurement portion 13, an activity amount calculation portion 14, a communication portion 15, a storage portion 16, and a display portion 17.

The body temperature measurement portion 11 measures the user's body temperature. The heart rate measurement portion 12 measures the heart rate per unit time of the user. The blood pressure measurement portion 13 measures the user's blood pressure. As long as the body temperature measurement portion 11, the heart rate measurement portion 12, and the blood pressure measurement portion 13 can effectively measure the user's body temperature, heart rate, and blood pressure, any measuring methods may be used. The body temperature measurement portion 11 measures a measurement value of 0 to 50° C., the heart rate measurement portion 12 measures a measurement value of 0 to 200 beats per minute, and the blood pressure measurement portion 13 measures a measurement value of 0 to 300 mm Hg. The body temperature measurement portion 11, the heart rate measurement portion 12, and the blood pressure measurement portion 13 may perform continuous measurement or may measure at predetermined time intervals (e.g., every minute or hour).

The activity amount calculation portion 14 calculates the activity amount (the number of steps, the amount of exercise (energy consumption), etc.) of the user. The activity amount calculation portion 14 includes sensors such as an acceleration sensor and a global positioning system (GPS) sensor, and calculates the number of steps and the amount of exercise based on the information obtained from these sensors. The activity amount calculation portion 14 also calculates the sleep duration of the user based on the acceleration sensor and the heart rate.

The communication portion 15 wirelessly communicates with a communication portion 21 of the mobile terminal device 20 to send the mobile terminal device 20 information such as the measurement values measured by the body temperature measurement portion 11, the heart rate measurement portion 12, and the blood pressure measurement portion 13, and the activity amount and sleep duration calculated by the activity amount calculation portion 14. The information sent by the communication portion 15 also includes time information indicating the time at which each of the measurement values, exercise amount, and sleep duration was obtained.

The storage portion 16 stores information such as the measurement values measured by the body temperature measurement portion 11, the heart rate measurement portion 12, and the blood pressure measurement portion 13, and the activity amount and sleep duration calculated by the activity amount calculation portion 14, for example. The information stored by the storage portion 16 also includes the time information indicating the time at which each of the measurement values, exercise amount, and sleep duration was obtained. The communication portion 15 sends the information stored in the storage portion 16 to the mobile terminal device 20.

The display portion 17, which is a display such as a liquid crystal display or an organic EL display, displays the measurement values measured by the body temperature measurement portion 11, the heart rate measurement portion 12, and the blood pressure measurement portion 13, the activity amount and the sleep duration calculated by the activity amount calculation portion 14, and the times, for example.

The mobile terminal device 20 includes the communication portion 21, a storage portion 22, a display portion 23, and a control portion 30.

The communication portion 21 receives information such as measurement values, activity amount, and sleep duration sent from the communication portion 15 of the wearable device 10, and sends the received information to the control portion 30 and the storage portion 22. The storage portion 22 stores the information sent from the communication portion 21 and data and programs processed by the control portion 30, for example. The display portion 23, which is a touch panel display such as a liquid crystal display or an organic EL display, displays an image transmitted from the control portion 30, receives an operation by the user, and sends a signal corresponding to the received operation to the control portion 30. The storage portion 22 stores an application of a program to be executed by the control portion 30, which will be described below, and the display portion 23 displays an icon A corresponding to the application.

The control portion 30 may be a central processing unit (CPU), for example, and includes an obtaining portion 31, a reference value determination portion 32, a health information creation portion 33, an analysis portion 34, an abnormality information determination portion 35, an option control portion 36, an improvement information control portion 37, and a day-of-the-week control portion 38.

The obtaining portion 31 obtains a plurality of types of information on the health of the user from the wearable device 10. Specifically, the obtaining portion 31 obtains information such as measurement values and an activity amount from the wearable device 10. This information includes information on the measurement values measured by the body temperature measurement portion 11, the heart rate measurement portion 12, and the blood pressure measurement portion 13 immediately before the obtainment, and information stored in the storage portion 16. For example, the information obtained from the storage portion 16 may be the information measured in the past week. The obtaining portion 31 may obtain such information at the time when the user touches the icon A to start the application, or when the user wants to check his or her health condition after starting the application and touches an icon for checking the health condition.

The reference value determination portion 32 determines a reference value used to determine whether the user's health condition is abnormal. For example, the reference value of body temperature may be the average value of body temperatures in the past week measured at about the same time as the current time. Alternatively, the reference value may be the average value of body temperatures in the past week measured at a predetermined time instead of the current time, or the average value of all body temperatures measured in the past week may be calculated as the reference value. As will be described below, the reference value may be calculated using only the body temperatures measured on the days of the week selected by the user. The reference value for blood pressure may be calculated in a similar manner. The systolic blood pressure (maximum blood pressure) may be measured as the blood pressure. Additionally, the storage portion 22 stores the amount of exercise in one week and the daily sleep duration that are considered ideal based on the age and other factors, and these amount of exercise and sleep duration are used as the reference values. The reference values of the amount of exercise and sleep duration may also be set by the user.

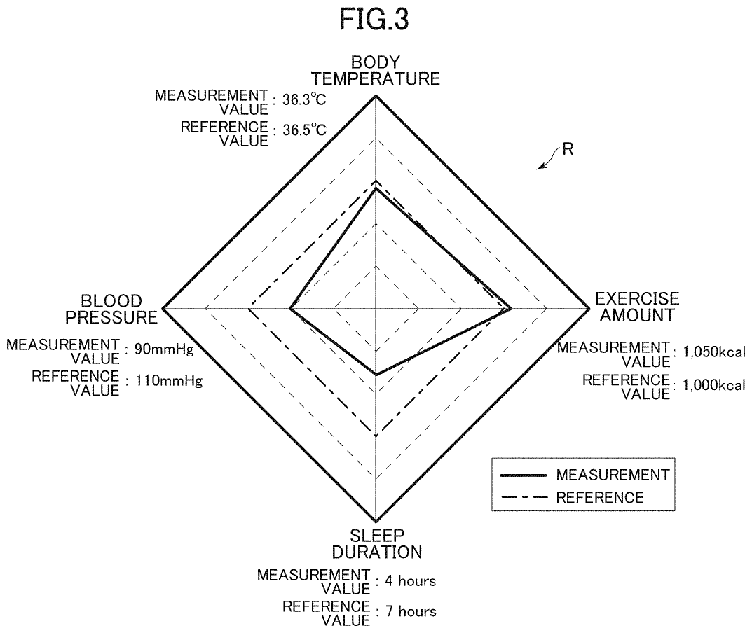

The health information creation portion 33 creates health information on the health condition of the user based on the information obtained by the obtaining portion 31 and causes the display portion 23 to display the health information. The health information may be a radar chart R regarding the health condition of the user illustrated in FIG. 3. The radar chart R shows the results of comparison between the measurement values and the reference values for body temperature, blood pressure (systolic blood pressure), sleep duration, and exercise amount.

As the measurement values, the current body temperature, the current blood pressure, the sleep duration of the previous day, and the amount of exercise in the past week are used. The user can ascertain his or her current health condition by glancing at the radar chart R. As illustrated in FIG. 3, the measurement values and the reference values of body temperature, blood pressure, sleep duration, and exercise amount may be displayed together with the radar chart R.

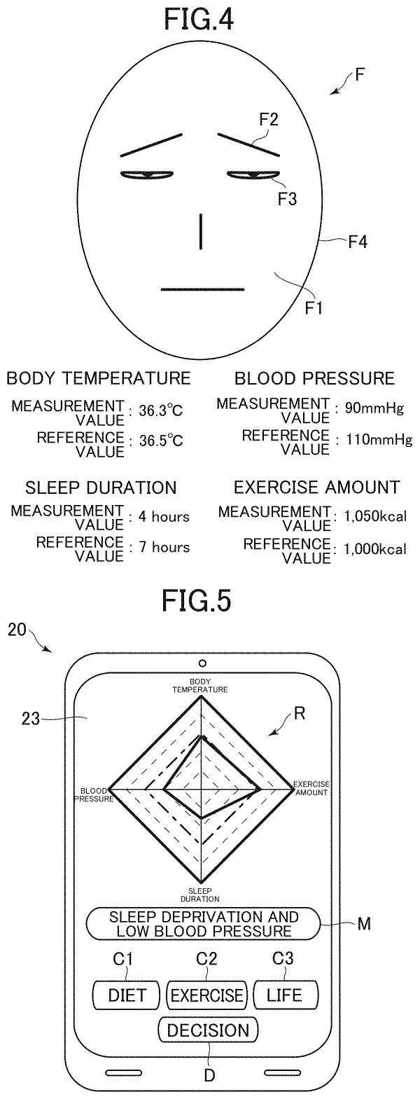

Furthermore, the health information on the health condition of the user may be an icon F of a human face as illustrated in FIG. 4, instead of the radar chart R of FIG. 3. The icon F shows the results of comparison between the reference values and the measurement values. The color of the face F1 indicates the body temperature, the angle of the eyebrows F2 indicates the blood pressure, the opening degree of the eyes F3 indicates the sleep duration, and the outline of the face F4 indicates the amount of exercise. As illustrated in FIG. 4, the measurement values and the reference values of body temperature, blood pressure, sleep duration, and exercise amount may be displayed together with the icon F.

As for the body temperature, the color of the face F1 may be peach when the current body temperature is equivalent to the reference value, red when higher than the reference value, and blue when lower than the reference value. As for the blood pressure, the angles of the eyebrows F2 are set such that the eyebrows F2 are horizontal when the current blood pressure is equivalent to the reference value, the outer ends are higher (tilted upward) when higher than the reference value, and the outer ends are lower (tilted downward) when lower than the reference value. As for the sleep duration, the eyes F3 open normally when the sleep duration on the previous day is equivalent to the reference value, the eyes F3 are wide open when longer than the reference value, and the eyes F3 are half closed when shorter than the reference value. As for the amount of exercise, the outline of the face F4 is generally the same as the outline of the user's face when the amount of exercise in the past week is equivalent to the reference value, the outline of the face F4 is narrower when larger than the reference value, and the outline of the face F4 is wider when smaller than the reference value. In this manner, a part of the human face icon F changes according to the value of the corresponding type of information on the health. As a result, the user can ascertain his or her current health condition by glancing at the icon F.

The analysis portion 34 analyzes whether the user's health condition has an abnormality based on the information obtained by the obtaining portion 31. For example, the analysis portion 34 determines a body temperature lower than 36.0° C. as hypothermia, a body temperature of 36.8 to 37.0° C. as a slight fever, a body temperature of 37.0 to 38.5° C. as a fever, and a body temperature higher than 38.5° C. as a high fever, thus determining these body temperatures as abnormal. The analysis portion 34 also determines a blood pressure lower than 100 mm Hg as a low blood pressure and a blood pressure higher than 140 mm Hg as a high blood pressure, thus determining these blood pressures as abnormal. A sleep duration of less than 5 hours is determined as insufficient and abnormal. An amount of exercise of less than 500 kcal is determined as insufficient and abnormal.

When the analysis portion 34 determines that the user's health condition has an abnormality, the abnormality information determination portion 35 determines information on the abnormality and causes the display portion 23 to display a character string indicating the information on the abnormality. For example, with the health condition of the radar chart R illustrated in FIG. 3, the blood pressure is lower than 100 mm Hg and thus low, and the sleep duration is shorter than 5 hours and thus insufficient. Accordingly, “Low blood pressure” and “Sleep deprivation” are determined as information on the abnormalities, and the display portion 23 displays “Sleep deprivation and Low blood pressure” as a character string M indicating the information on the abnormalities. As illustrated in FIG. 5, the abnormality information determination portion 35 displays the character string M below the radar chart R. This display process is performed by combining the information on the abnormalities with a predetermined template.

The option control portion 36 displays a plurality of user-selectable option icons C1 to C3 to improve the information on an abnormality on the display portion 23. As illustrated in FIG. 5, the option control portion 36 displays Diet C1, Exercise C2, and Life C3 as option icons. The user can select at least one of Diet C1, Exercise C2, and Life C3. After selection, the icon of Dicision D is touched to display information to improve the information on the abnormality on the display portion 23.

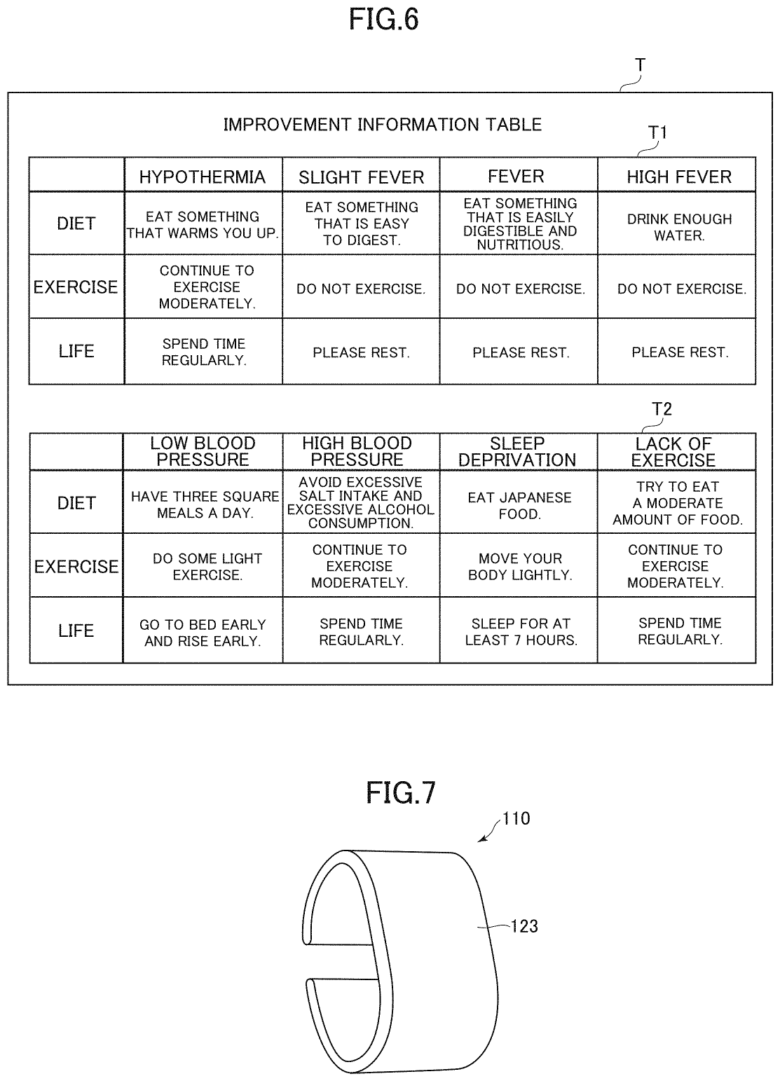

The improvement information control portion 37 displays the improvement information corresponding to the option icon selected by the user on the display portion 23. The improvement information corresponding to the option icon selected by the user is determined based on a improvement information table T illustrated in FIG. 6. The storage portion 22 stores this improvement information table T. The improvement information table T includes a first table T1 and a second table T2. The first table T1 is a table that associates “Hypothermia”, “Slight fever”, “Fever”, and “High fever” with improvement methods regarding “Diet”, “Exercise”, and “Life”. The second table T2 is a table that associates “Low blood pressure”, “High blood pressure”, “Sleep deprivation”, and “Lack of exercise” with improvement methods regarding “Diet”, “Exercise”, and “Life”.

For example, as illustrated in FIG. 5, when the abnormality information includes “Sleep deprivation” and “Low blood pressure” and the user selects Life C3, the display portion 23 displays “Go to bed early and rise early.” and “Sleep for at least 7 hours.” The improvement information control portion 37 may display such indications on the display portion 23 at a predetermined time. For example, “Go to bed early and rise early.” and “Sleep for at least 7 hours.” may be displayed on the display portion 23 at around 10 p.m. Also, when the abnormality information is “Low blood pressure” and the user selects Diet C1, the display portion 23 may display “Have three square meals a day.” at 6 a.m., 11:30 a.m., and 6 p.m.

The indications may also be displayed on the display portion 17 of the wearable device 10. Furthermore, when the wearable device 10 and/or the mobile terminal device 20 have an alarm function, indications may be displayed in conjunction with the alarm function.

The day-of-the-week control portion 38 causes the display portion 23 to display the day-of-the-week selection information for allowing the user to select a plurality of days of the week. A day-of-the-week selection icon is displayed, and when the user touches this icon, “Monday”, “Tuesday”, “Wednesday”, “Thursday”, “Friday”, “Saturday”, and “Sunday” are displayed on the display portion 23. This allows the user to select a day of the week that is not used to calculate a reference value. For example, when the user works from Monday to Friday and does not work on Saturday or Sunday, the user may select Saturday and Sunday because the values of body temperature and blood pressure on Saturday and Sunday may differ from those on Monday to Friday. Then, the reference value determination portion 32 calculates the reference values for body temperature and blood pressure without using the values measured on Saturday or Sunday. Alternatively, the user may select days of the week that are used for the calculation of reference values, and the reference values may be calculated using the values measured on the selected days of the week.

In the health assistance system 1 as described above, the health information creation portion 33 creates health information on the user's health condition based on the information obtained by the obtaining portion 31, and causes the display portion 23 to display the health information. The health information may be the radar chart R (FIG. 3) or the human face icon F (FIG. 4) regarding the user's health. This allows the user to ascertain his or her current health condition by glancing at the radar chart R or the human face icon F.

When the analysis portion 34 determines that the user's health condition has an abnormality, the abnormality information determination portion 35 determines information on the abnormality and causes the display portion 23 to display a character string indicating the information on the abnormality. As a result, the user can ascertain his or her current health condition also by reading the character string.

The option control portion 36 displays on the display portion 23 a plurality of user-selectable options C1 to C3 to improve the information on an abnormality. The options include a diet option regarding diet (Diet C1), an exercise option regarding exercise (Exercise C2), and a life option regarding lifestyle (Life C3). This allows the user to select a preferable improvement method.

The improvement information control portion 37 displays information on the option selected by the user (information to improve the abnormality) on the display portion 23 at a predetermined time. Accordingly, the user is prompted to take action to improve the abnormality at an appropriate time.

Based on the days of the week selected by the user, the reference value determination portion 32 calculates a reference value, which is to be used by the analysis portion 34 to determine whether the user's health condition has an abnormality, using the user's body temperature and blood pressure obtained by the obtaining portion 31. As such, the days of the week on which the life rhythm differs from the normal life rhythm can be excluded when calculating the reference value, thereby improving the accuracy of abnormality determination.

The present disclosure is not limited to the embodiments described above. A person skilled in the art can make various additions and modifications within the scope of the present disclosure.

For example, instead of the health assistance system 1, a wearable device 110 as illustrated in FIG. 7 may be used. This wearable device 110 includes all the configurations of the wearable device 10 and the mobile terminal device 20. The band-shaped wearable device 110 also includes a bendable organic EL touch display as a display portion 123, which substantially extends over its entire outer circumferential surface. The display portion 123 displays information such as the radar chart R regarding the user's health condition. The wearable device 110 does not have to include the display portion 17 and the storage portion 16 of the wearable device 10.

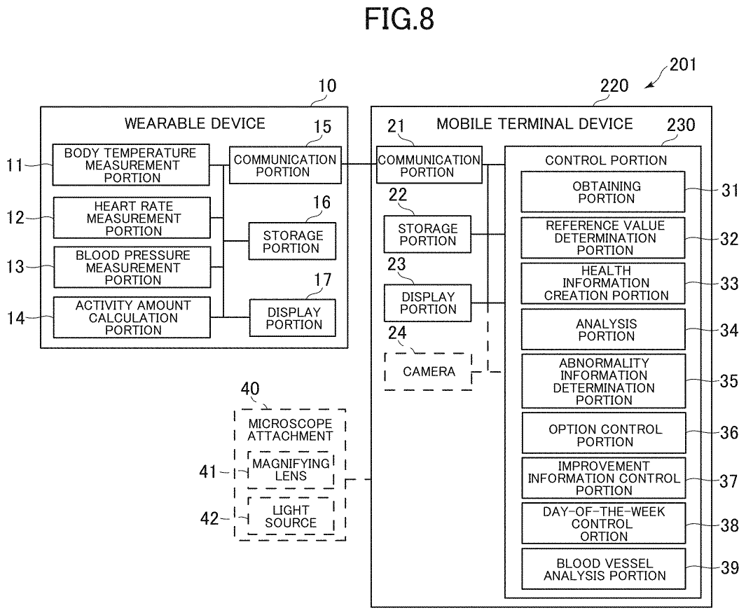

As illustrated in a health assistance system 201 of FIG. 8, a control portion 230 of a mobile terminal device 220 may include a blood vessel analysis portion 39. For example, a device capable of capturing images (still images and moving images) of blood vessels (capillaries) in a fingertip may capture an image of a blood vessel of the user's fingertip. The information on the blood vessel image may be sent to the wearable device 10 via short-range wireless communication and stored in the storage portion 16. Then, the blood vessel analysis portion 39 may analyze the blood vessel based on the information on the blood vessel image obtained by the obtaining portion 31 from the wearable device 10.

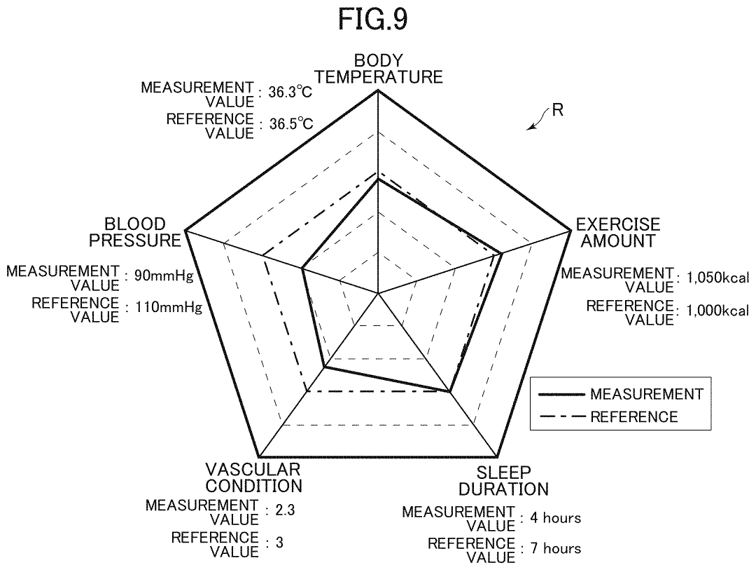

In this analysis, a plurality of types of vascular elements, such as length (μm), thickness (μm), number of bends (number of points at which the blood vessel bends at a predetermined angle or more)/mm), and sharpness (brightness), are analyzed to determine whether the blood vessel has an abnormality. The determination may be based comprehensively on all the vascular elements, or may be based on one or some of the elements. For example, when the sharpness (brightness) is less than the predetermined determination value, it is determined that the vascular condition is abnormal. As illustrated in FIG. 9, the radar chart R may include an axis regarding vascular information. In FIG. 9, the sharpness of a relatively healthy blood vessel is set to 3, which is used as the reference value of vascular condition. Furthermore, the improvement information table T may include a improvement method for an abnormality of “Vascular condition” so that the display portion 23 can display the improvement method.

Additionally, instead of providing the blood vessel analysis portion 39 in the control portion 230 of the mobile terminal device 220, the wearable device 10 may transmit a blood vessel image to a server device (not illustrated) having blood vessel analysis software, via a communication network such as the Internet. The wearable device 10 may receive an analysis result from this server device and send the analysis result to the mobile terminal device 220.

Furthermore, as indicated by broken lines in FIG. 8, the mobile terminal device 220 may include a camera 24 capable of capturing images, and a microscope attachment 40, which has a magnifying lens 41 and a light source 42, may be attached to the camera 24. An image of a blood vessel in the user's fingertip may be captured, and the blood vessel analysis portion 39 may analyze the vascular condition.

In addition to body temperature, blood pressure, and vascular condition, information such as saliva, oral condition, sweating amount, urine component, and blood may be obtained and analyzed. Furthermore, such information may be measured by the wearable device 10, 110 or the mobile terminal device 20 itself, or the wearable device 10, 110 or the mobile terminal device 20 may obtain information measured by another device.

What is claimed is:

1. A health assistance system comprising:
a display portion;
an obtaining portion that is configured to obtain a plurality of types of information on health of a user; and
a health information creation portion that is configured to create health information on a health condition of the user, based on the information obtained by the obtaining portion, and cause the display portion to display the health information.

2. The health assistance system according to claim 1, wherein the health information displayed on the display portion is a radar chart or a human face icon.

3. The health assistance system according to claim 2, wherein a part of the human face icon is configured to be changed according to a value of a corresponding one of the types of information on the health.

4. The health assistance system according to claim 1, further comprising:
an analysis portion that is configured to analyze whether the health condition of the user has an abnormality based on the information obtained by the obtaining portion; and
an abnormality information determination portion that is configured to, in response to the analysis portion determining that the health condition of the user has an abnormality, determine information on the abnormality and cause the display portion to display a character string indicating the information on the abnormality.

5. The health assistance system according to claim 4, further comprising an option control portion that is configured to cause the display portion to display a plurality of user-selectable options to improve the information on the abnormality,
wherein the options include a diet option regarding diet, an exercise option regarding exercise, and a life option regarding lifestyle.

6. The health assistance system according to claim 5, further comprising a improvement information control portion that is configured to cause the display portion to display information on the option selected by the user at a predetermined time.

7. The health assistance system according to claim 4, wherein the plurality of types of information on the health of the user includes information on a body temperature and a blood pressure of the user,
the health assistance system further comprising:
a day-of-the-week control portion that is configured to cause the display portion to display day-of-the-week selection information for allowing the user to select a plurality of days of week; and
a reference value determination portion that is configured to, based on the days of the week selected by the user, calculate a reference value that is to be used by the analysis portion to determine whether the health condition of the user has an abnormality, by using the body temperature and the blood pressure of the user obtained by the obtaining portion.

8. A wearable device comprising:
a display portion;
an obtaining portion that is configured to obtain a plurality of types of information on health of a user; and
a health information creation portion that is configured to create health information on a health condition of the user based on the information obtained by the obtaining portion, and cause the display portion to display the health information.
